(19) United States
(12) Patent Application Publication
(10) Pub. No.: US 2017/0245792 A1
(43) Pub. Date: Aug. 31, 2017

(54) Skin Test Reading Device and Associated Systems and Methods

(71) Applicant: Flare Diagnostics, LLC, West Friendship, MD (US)

(72) Inventors: Jody Robert TVERSKY, West Friendship, MD (US); Donald Welton MACGLASHAN, JR., Middle River, MD (US)

(73) Assignee: Flare Diagnostics, LLC, West Friendship, MD (US)

(21) Appl. No.: 15/519,910

(22) PCT Filed: Oct. 20, 2015
(86) PCT No.: PCT/US15/56330

§ 371 Date: Apr. 18, 2017

Related U.S. Application Data
(60) Provisional application No. 62/066,349, filed on Oct. 20, 2014.

(51) Int. Cl. A61B 5/00; G06T 7/00

(52) U.S. Cl. CPC A61B 5/411; A61B 5/742; A61B 5/0077; A61B 5/0075; G06T 7/0012; G06T 2207/10048; G06T 2207/30088

(57) ABSTRACT

Exemplary embodiments are directed to skin test reading devices that include a housing and a short wave infrared (SWIR) detector. The SWIR detector can include a lens configured to enable the SWIR detector to capture an image of a skin testing area of a patient. The SWIR detector can be configured to detect a wheal formed on the skin testing area of the patient. Exemplary embodiments are also directed to methods of reading a skin test and skin test reading systems.

### CROSS-REFERENCE TO RELATED APPLICATIONS

This application claims the benefit of co-pending, commonly assigned U.S. Provisional patent application entitled “Automated Allergy Skin Test Reading Device” which was filed on Oct. 20, 2014, and assigned Ser. No. 62/066,349. The entire contents of the foregoing provisional patent application are incorporated herein by reference.

### TECHNICAL FIELD

The present disclosure relates to skin test reading devices and, in particular, to automated skin test reading devices that diagnose allergic disease and skin disorders in an efficient, precise and consistent manner.

### BACKGROUND

In the medical industry, skin tests are performed for various purposes, e.g., to diagnose allergies of a patient. The skin tests involve introduction of one or more allergens to the skin of the patient with a needle, pin or syringe by various methods, such as pricking, scratching, puncturing or intradermally. Immuno-responses seen in the form of a wheal (e.g., a red, swollen or raised mark) and flare (e.g., burn) on the patient's skin indicate a hypersensitivity or allergy to the allergen.

In general, the immuno-responses are manually detected, read and analyzed by a technician. Manual reading of the wheal or flare result can be laborious and is often inaccurate due to human error. In particular, taking measurements of the size and extent of the wheal or flare can be a tedious and time-consuming process. For example, erythema (e.g., flare) coloration can be faint and nearly unreadable or undetectable in dark-skinned patients. Measurement of the diameter of each wheal is typically manually performed one-by-one with a ruler, which can result in inaccurate measurements, and the diameter of the wheal can be a poor predictor of the true area that is raised on the skin. Errors can occur due to poor technique or complex labeling of the testing area as well. For example, when 40-50 skin prick tests are placed on a patient in the setting of a busy clinic, errors in labeling and/or transferring results can occur. Patient discomfort from the itch can be prolonged due to the extensive time used for manual readings. In addition, reproducibility of the test generally cannot be achieved and can be suboptimal. A lack of standardization in testing and reading the wheal or flare due to user variation can negatively impact research. The inability to capture results or images of the test electronically can reduce the effectiveness of laboratory records, and manual data entry of the results into an electronic medical record increases the already time-consuming process.

To accommodate the issues associated with the typical practice described above, clinicians have used more gross interpretation of the wheal and, in some cases, ignore the flare altogether. The impact of discarding such important data is unknown. In particular, food and drug allergy testing often necessitates a high level of precision to determine if patients may have a strong reaction to an allergen. Therefore, unreliable interpretation of the allergy skin tests can have dramatic consequences.

Thus, a need exists for automated devices for accurately determining the immune-responses of a patient to allergy tests in an efficient manner. These and other needs are addressed by the skin test reading devices and associated systems and methods of the present disclosure.

### SUMMARY

In accordance with embodiments of the present disclosure, an exemplary skin test reading device is provided that includes a housing and a short wave infrared (SWIR) detector which may be at least partially disposed within the housing. The SWIR detector can include a lens configured to enable the SWIR detector to capture one or more images of a skin testing area of a patient. The SWIR detector can be configured to detect a wheal formed on the skin testing area of the patient during an allergen test in the captured image.

In some embodiments, the SWIR detector can include a filter configured to isolate or remove predetermined wavelengths of light associated with the skin testing area. In some embodiments, the filter can be configured to obscure veins in the testing area to enhance detection and visibility of the wheal formed on the skin testing area of the patient. In some embodiments, the SWIR detector can be configured to detect wavelengths between approximately 1,000 nm and approximately 3,000 nm.

In some embodiments, the device can include a near wave infrared (NIR) detector at least partially disposed within the housing. The NIR detector can include a lens configured to enable the NIR detector to capture an image of the wheal formed on the skin testing area of the patient. In some embodiments, the NIR detector can be configured to detect wavelengths between approximately 700 nm and approximately 1,000 nm.

In some embodiments, the device can include a thermal infrared detector at least partially disposed within the housing. The thermal infrared detector can include a lens configured to enable the thermal infrared detector to capture an image of the skin testing area of the patient. In particular, the thermal infrared detector can be configured to detect and display a flare formed on the skin testing area of the patient in the captured image during the allergen test. The flare can be associated with the wheal detected by the SWIR detector. In some embodiments, the thermal infrared detector can be configured to detect wavelengths between approximately 7,000 nm and approximately 14,000 nm.

In some embodiments, the device can include a visual wavelength detector at least partially disposed within the housing. The visual wavelength detector can include a lens configured to enable the visual wavelength detector to capture an image of the skin testing area of the patient. In some embodiments, the visual wavelength detector can be configured to detect wavelengths between approximately 400 nm and approximately 700 nm.

The device can include a processing device disposed within the housing. The processing device can be configured to analyze the wheal and/or flare and output a dimension associated with the wheal and/or flare. In some embodiments, the dimension of the wheal can be a wheal diameter or a wheal area. In some embodiments, the device can include a graphical user interface (GUI) configured to display the captured and/or processed image of the wheal, flare, or both, and the associated data to a user.

In accordance with embodiments of the present disclosure, an exemplary method of reading a skin test is provided. The method includes providing a skin test reading device as disclosed herein. The method includes directing the SWIR detector at a skin testing area of a patient. The method further includes capturing one or more images of the skin testing area of the patient with the lens of the SWIR detector. The SWIR detector can be configured to detect the wheal formed on the skin testing area of the patient.

In some embodiments, the method can include filtering specific or predetermined wavelengths captured by the SWIR detector with a filter disposed within the SWIR detector to isolate or remove predetermined wavelengths of light associated with the skin testing area, thereby enhancing visibility of the wheal. In some embodiments, the method can include directing a NIR detector at the skin testing area of the patient and capturing an image of the skin testing area of the patient with a lens of the NIR detector. The NIR detector can be configured to detect and display the wheal formed on the skin testing area of the patient in the captured image.

In some embodiments, the method can include directing a thermal infrared detector at the skin testing area of the patient and capturing an image of the skin testing area of the patient with a lens of the thermal infrared detector. The thermal infrared detector can be configured to detect and display a flare formed on the skin testing area of the patient in the captured image. In some embodiments, the method can include programmatically and electronically transmitting the one or more captured images to an electronic medical file or record.

In accordance with embodiments of the present disclosure, an exemplary skin test reading system is provided that includes a skin test reading device as disclosed herein. The system can include a supporting structure configured to mount the SWIR detector (or alternative/additional detectors) over the skin testing area of the patient. The system can include one or more illumination devices mounted to the supporting structure. The illumination devices can be configured to illuminate at least a portion of the skin testing area of the patient with predetermined wavelengths of light. In some embodiments, the illumination devices can illuminate the skin testing area with wavelengths that enhance the SWIR and/or thermal visibility of the wheal and/or flare. The system can include a graphical user interface configured to display the image captured by the SWIR detector (or alternative/additional detectors).

In accordance with embodiments of the present disclosure, an exemplary automated allergen skin test reading device is provided. In some embodiments, the device can include a SWIR imaging camera for detection of a wheal (e.g., a raised area on the skin of the patient). In some embodiments, the device can include a thermal imaging camera for detection of a flare (e.g., an erythematous area on the skin of the patient). In some embodiments, the device can include a combination of the SWIR imaging camera and the thermal imaging camera. The device can include a processing device configured to process a software algorithm for processing a captured skin test image. The device can include the appropriate hardware components required to connect the elements of the device and facilitate communication between the elements.

In some embodiments, the device can include a NIR imaging camera for detection of the wheal. The device can be configured for attachment to and/or communication or interfacing with a processing device, e.g., a computer, a laptop, a handheld device, combinations thereof, or the like. In some embodiments, the device can be configured for connection and/or interfacing directly or indirectly with a clinical or hospital-based electronic medical record such that collected data can be transmitted to and stored in the clinical or hospital-based electronic medical record. In some embodiments, the device can be configured for connection and/or interfacing directly or indirectly with a research database such that collected data can be transmitted to and stored in the research database.

The device can utilize a specific bandpass or longpass filter to isolate or subtract specific wavelengths of light in order to enhance the water content in the skin. In some embodiments, the device can include software incorporated therein that permits user input for defining labeling for identification of the allergen and control substances tested. The device can be optimized to measure the wheal and/or flare responses in combination with skin prick devices. The device can be optimized to measure the wheal and/or flare responses following, e.g., intradermal testing, the subcutaneous injection of allergen immunotherapy, the injection of vaccines, the injection of biologics, combinations thereof, or the like.

In some embodiments, the device can include software configured to permit adjustment of the components to scan larger surface areas of the tested region to detect and measure urticarial (e.g., hive) lesions on the skin. In some embodiments, the device can be optimized to detect erythema, hives, and flushing associated with anaphylaxis. As discussed herein, the device can be optimized to measure wheal and/or flare responses on the skin of humans. In particular, the device can be optimized to measure wheal and/or flare responses of dark-skinned individuals in an accurate manner. The device can be optimized to measure wheal and/or flare responses on the skin of, e.g., the forearm, back, animals, humans, or the like.

Other objects and features will become apparent from the following detailed description considered in conjunction with the accompanying drawings. It is to be understood, however, that the drawings are designed as an illustration only and not as a definition of the limits of the invention.

### BRIEF DESCRIPTION OF THE DRAWINGS

To assist those of skill in the art in making and using the disclosed devices and associated systems and methods, reference is made to the accompanying figures, wherein:

FIG. 1 is a diagrammatic view of an exemplary skin test reading system in accordance with embodiments of the present disclosure;

FIG. 2 is a diagram of an electromagnetic spectrum;

FIG. 3 is a block diagram of an exemplary skin test reading system in accordance with embodiments of the present disclosure;

FIG. 4 is a block diagram of an exemplary skin test reading system in accordance with embodiments of the present disclosure;

FIG. 5 is a block diagram of a computing device configured to implement embodiments of an exemplary skin test reading system in accordance with embodiments of the present disclosure;

FIG. 6 is a block diagram of a distributed environment for implementing embodiments of an exemplary skin test reading system in accordance with embodiments of the present disclosure;

FIG. 7 is a perspective view of an exemplary prototype of a skin test reading device in accordance with embodiments of the present disclosure;

FIG. 8 is a detailed, perspective view of a prototype of an exemplary SWIR detector of a skin test reading device in accordance with embodiments of the present disclosure;

FIG. 9 is a detailed, perspective view of a prototype of an exemplary illumination device of a skin test reading device in accordance with embodiments of the present disclosure;

FIG. 10 is a flow chart of an exemplary process of reading a skin test with an exemplary skin test reading device in accordance with embodiments of the present disclosure;

FIG. 11 is a flow chart of an exemplary process of reading a skin test with an exemplary skin test reading device in accordance with embodiments of the present disclosure;

FIG. 12 is an image of a skin testing area of a patient captured by a visual detector in accordance with embodiments of the present disclosure;

FIG. 13 is an image of a skin testing area of a patient captured by a NIR detector in accordance with embodiments of the present disclosure;

FIG. 14 is an image of a skin testing area of a patient captured by a thermal infrared detector in accordance with embodiments of the present disclosure;

FIG. 15 is an image of a skin testing area of a patient captured by a SWIR detector in accordance with embodiments of the present disclosure;

FIG. 16 is an image of a skin testing area of a patient captured by a SWIR detector in accordance with embodiments of the present disclosure;

FIG. 17 is a processed image of a skin testing area of a patient captured by a SWIR detector in accordance with embodiments of the present disclosure;

FIG. 18 is an image of a skin testing area of a patient captured by a thermal infrared detector in accordance with embodiments of the present disclosure; and

FIGS. 19A-G are images of a skin testing area of a patient captured by a SWIR detector and a thermal infrared detector in accordance with embodiments of the present disclosure.

### DESCRIPTION OF EXEMPLARY EMBODIMENTS

In accordance with embodiments of the present disclosure, an exemplary automated skin test reading device is provided. The device can be implemented in allergy or dermatology settings. In particular, the device can be used to diagnose allergic disease and/or skin disorders, such as allergic rhinitis, food allergy, asthma, urticaria, contact allergies, psoriasis, or the like.

The devices can capture and analyze images of skin testing during which allergens, histamine and/or control substances are placed on the skin of a patient. Although discussed herein with respect to testing of human patients, it should be understood that in some embodiments, the devices can be used for testing of animal patients. Thousands of allergens can be tested in practice including aeroallergens (e.g., dust mites, pollen, animal dander, or the like) and foods (e.g., peanut, shellfish, egg, wheat, dairy, or the like). Allergens tested can also include drugs and biologics, such as antibiotics. Venoms, such as wasp or hornet, can also be tested. In addition, any substance or protein can be tested on the skin with the typical skin testing methods, and the devices discussed herein can be used to accurately and consistently analyze the results.

The device includes various elements for detecting and measuring the wheal and/or flare response for allergen skin testing. The device can include, e.g., a visible detector (which may include color), a short range or wave infrared (SWIR) detector, a near range or wave infrared (NIR) detector, a thermal infrared detector, combinations thereof, or the like, along with a processing device configured to execute customized image processing instructions or algorithms to accurately detect skin test responses. The device operates effectively and accurately by measuring responses on the skin of any individual regardless of skin pigmentation and other physiological variability. The accuracy and efficiency of the device can meet the specific needs of clinicians and researchers who investigate allergic and dermatological conditions in humans and animals worldwide.

The system associated with the device can include optimal detectors, lighting and hardware components, as well as precise software elements for analyzing the captured images and data. The devices can be configured to use specific wavelengths of light to measure wheal and/or flare reactions more accurately. For example, the SWIR detector can measure the wheal response in part by isolating water content in the skin that would not otherwise be visible to the human eye, thereby accurately and precisely measuring the wheal response of a patient. In particular, data from the SWIR detector can be used to calculate the diameter and/or area of the wheal by way of an automated imaging software. In some embodiments, a thermal infrared detector can be used individually, sequentially or simultaneously with the SWIR detector to capture and measure flare responses of the patient, thereby accurately and precisely measuring the flare response of a patient.

The system can include a variety of controls for operate/controlling the disclosed devices. In some embodiments, the devices can include manual and/or automated control of zoom, focus, tiling and mounting solutions. The captured images can be controlled and analyzed by a software interface associated with a processing device of the system. The software interface can include digital control of the image area, sensitivity, gain, aperture, exposure time, or the like, to enhance the captured images. Incoming data can also be labeled by a user through the system such that each wheal and/or flare response reading can be matched to a user-defined skin testing template.

Various computer algorithm and processing software can be incorporated into a processing device for analyzing the captured images. For example, the digital information obtained from the SWIR and thermal infrared detectors can be displayed directly on a graphical user interface (e.g., a monitor) for visualization by a user. The automated processing can include an analysis of the images that outputs the diameter and area of the wheal and/or flare in just a few seconds. In some embodiments, the image processing algorithm can include a series of transformations, such as a smoothing and convolution filter, contrast improvement, binary thresholding, outline, or the like.

In some embodiments, the device can be programmatically configured to electronically report and archive the skin test results. In some embodiments, the device can be programmatically configured to electronically transmit, incorporate and archive the skin test results in an electronic medical record (EMR). The data associated with the skin test results can include captured images by the detectors of the device and the resulting analysis, thereby providing a means of storing images of the test in a patient medical record. The ability to automate the allergen skin test measurements and store an archived image of the results has the potential to reduce errors and can save lives of patients. The stored images can be retrieved at a later date for verification and for research purposes.

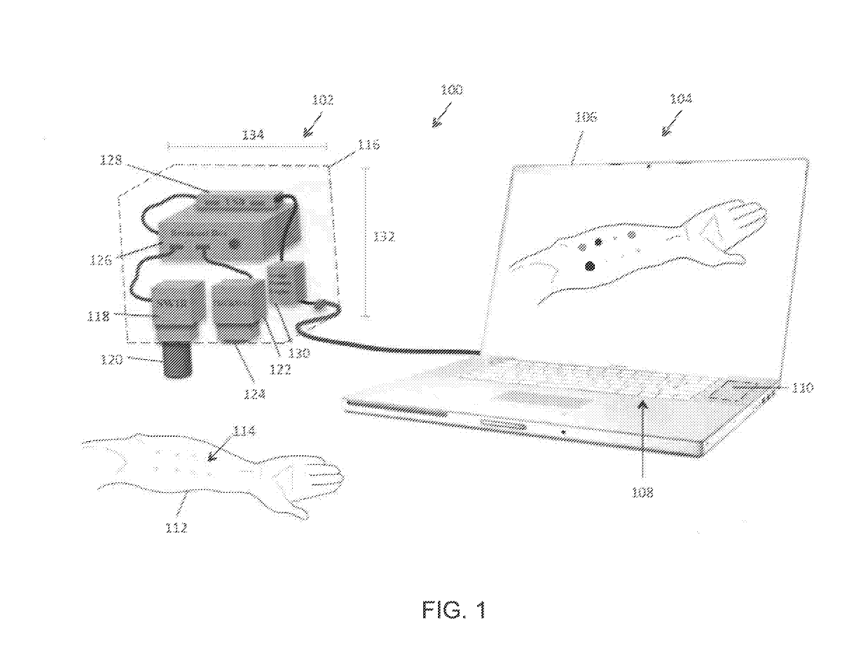

Turning now to FIG. 1, a diagrammatic view of an exemplary skin test reading system 100 (hereinafter “system 100”) is provided. The automated system 100 can be used in clinical practice or research to detect and measure wheal and flare of a patient, e.g., human, animal, or the like, during allergy testing. As an example, the system 100 can be used in dermatology and otolaryngology applications for detecting and measuring hives, skin infections, immunotherapy reactions, anaphylaxis, patch testing, or the like.

The system 100 can include an exemplary skin test reading device 102 (hereinafter “device 102”) in communication with a user interface 104. The device 102 can be connected to the user interface 104 in a wired and/or wireless manner such that data can be transmitted therebetween. For example, the device 102 can be connected to the user interface 104 via a wire or cable, as shown in FIG. 1. In some embodiments, the user interface 104 can include a graphical user interface (GUI) 106 (e.g., a display) and an input interface 108 (e.g., a keyboard, mouse, or the like). The GUI 106 can be used to display images and/or data to a user. The input interface 108 can be used to input data or instructions into the user interface 104. The user interface 104 can include a processing device 110 for receiving/transmitting data from the device 102, and for processing the received data. In some embodiments, the user interface 104 can be a desktop computer, a laptop computer, or a handheld device.

The system 100 can be used to measure the wheal and flare response on humans and/or animals following allergen skin testing. As an example, a diagram of an arm 112 of a patient is provided in FIG. 1 with a plurality of testing areas 114 for an allergy skin prick test. Although discussed herein with respect to testing of an arm 112 of a patient, it should be understood that the system 100 can be used for testing various anatomical locations of the human or animal body.

The device 102 can include a housing 116 configured and dimensioned to enclose or partially enclose components of the device 102. Although illustrated as disposed within the housing 116, it should be understood that some or portions of the components can be disposed outside of the housing 116. In some embodiments, the housing can define a height 132 and a width 134. In some embodiments, the height 132 and width 134 can be dimensioned as approximately 12 cm. In some embodiments, a GUI can be incorporated into the device 102 to display a digital representation of captured images and/or data. The device 102 can include a short wave infrared (SWIR) detector 118 at least partially disposed within the housing 116. In some cases, the SWIR detector may be fabricated to also enable detection of NIR and visible ranges. The SWIR detector 118 can include a camera therein with a lens 120 for capturing an image of one or more testing areas 114 of the patient. In some embodiments, the lens 120 can be actuated to zoom in or out to capture the desired area of the patient. For example, the lens 120 can be zoomed out to capture the entire testing region of the patient (e.g., the eight testing areas 114 shown in FIG. 1) or can be zoomed in to capture one or more specific testing areas 114. The SWIR detector 118 can include a filter disposed therein and associated with the lens 120.

In some embodiments, the device 102 can include one or more additional detectors 122 with a lens 124. For example, the detector 122 can be a visible (which can include color) detector, a near range or wave infrared (NIR) detector, a thermal infrared detector, or longwave infrared (LWIR) detector, combinations thereof, or the like. A visible detector can provide an image of the testing areas 114 using visible electromagnetic wavelengths. A NIR detector can delineate fine detail and contrast outside of the visible spectrum. A thermal infrared detector can use thermal radiation wavelengths to detect and measure flare associated with the testing areas 114.

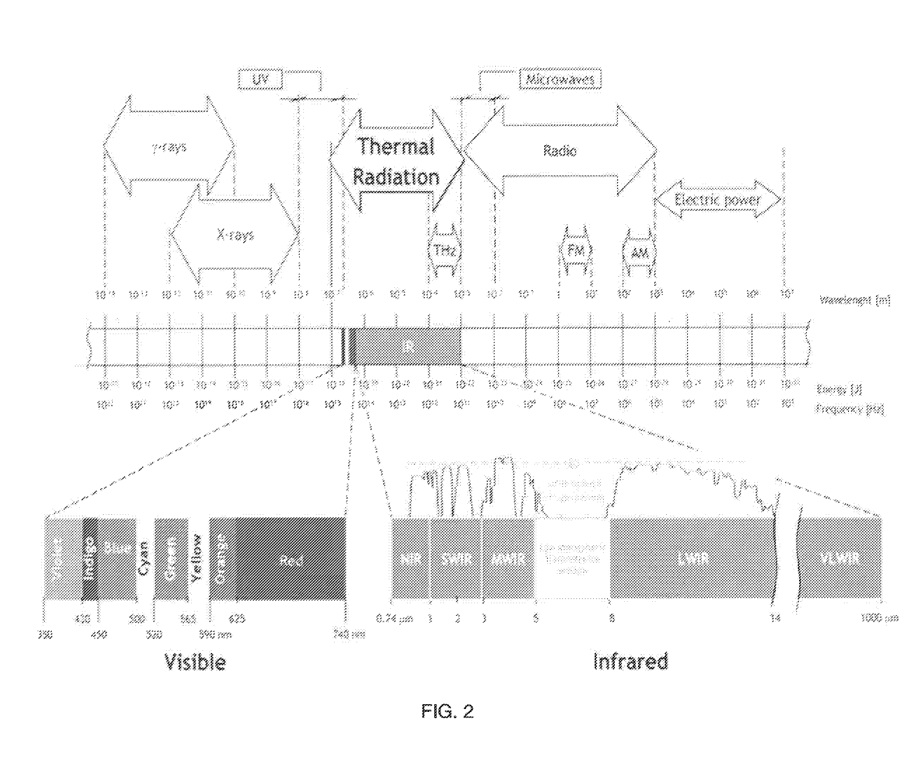

With reference to FIG. 2, a diagram of an electromagnetic spectrum is provided. The visible spectrum can range from approximately 350 nm to 740 nm. At wavelengths longer than the visible red light that human eyes can see is the portion of the spectrum generally described as the infrared region. Infrared light ranges from approximately a 700 nm wavelength to approximately a 1,000 μm (1,000,000 nm) wavelength, and can be subdivided into specific subset regions. NIR wavelengths detected can range from approximately 700 nm to approximately 1,000 nm (although detectors optimized for NIR can span the visible spectrum as well). In some embodiments, the NIR detector can detect wavelengths between, e.g., approximately 700 nm and approximately 1,000 nm, approximately 700 nm and approximately 950 nm, approximately 700 nm and approximately 900 nm, approximately 700 nm and approximately 850 nm, approximately 700 nm and approximately 800 nm, approximately 750 nm and approximately 1,000 nm, approximately 800 nm and approximately 1,000 nm, approximately 850 nm and approximately 1,000 nm, approximately 900 nm and approximately 1,000 nm, approximately 750 nm and approximately 950 nm, approximately 800 nm and approximately 900 nm, or the like.

SWIR camera detectors generally are made with semiconductor materials that absorb light in the full range of 700 to 3000 nm, or various sub-ranges depending on formulation. Examples are the compound semiconductors of indium gallium arsenide (InGaAs), Indium antimonide (InSb), mercury cadmium telluride (HgCdTe also known as MCT), silicon-germanium (SiGe), or silicon doped with sulphur, sometimes referred to as black silicon. SWIR detectors can detect wavelengths between, e.g., approximately 1,500 nm and approximately 2,500 nm, approximately 1,100 nm and approximately 2,200 nm, approximately 1,000 nm and approximately 3,000 nm, approximately 1,000 nm and approximately 2,500 nm, approximately 1,000 nm and approximately 2,000 nm, approximately 1,000 nm and approximately 1,800 nm, approximately 1,000 nm and approximately 1,600 nm, approximately 1,200 nm and approximately 2,500 nm, approximately 1,200 nm and approximately 2,000 nm, approximately 1,200 nm and approximately 1,800 nm, approximately 1,200 nm and approximately 1,600 nm, approximately 1,350 and approximately 1,800 nm, approximately 1,800 nm and approximately 1,600 nm, approximately 1,350 nm and approximately 1,450 nm, approximately 1850 nm and approximately 1950 nm, or the like. SWIR detectors can be most sensitive in the, e.g., approximately 1,000 nm to approximately 2,500 nm, approximately 1,000 nm and approximately 1,800 nm, approximately, approximately 1,350 nm and approximately 1,450 nm, or the like, and combinations thereof wavelength range.

SWIR detection systems can detect contrast in reflection from skin based on different water molecule concentration due to water absorption of light in certain wavelength bands. Optical bandpass filters or illumination sources that restrict the wavelengths of light detected by the SWIR detection system can be used to isolate objects or regions of tissue with more water from surrounding surfaces or tissue. In some embodiments, one or more physical and/or digital filters can be used to enhance the captured images. For example, a mosaic pattern of filters on the SWIR detector, or alternate means of encoding some pixels with response in one wavelength band and others to other wavelength bands, could permit simultaneous capture of images in two or more bands. Software algorithms could process the resultant data to make more accurate determination of the wheal boundaries. In some embodiments, the SWIR detector can include one or more filters for focusing on the approximately 1,350 nm to approximately 1,450 nm range. In some embodiments, one or more physical and/or digital filters can be used to enhance the captured images. Specific regions of the skin can be resolved with greater detail in part due to the differences in spectral reflection, depth of tissue, and water content. This is essential when imaging a wheal located on the skin, since wheal formation is characterized by increased water content compared to surrounding tissue. Thus, the advantageous properties of the SWIR detector 118 allow for accurate detection and imaging of wheals in the testing area 114.

The SWIR detector 118 can be used to resolve or block water vapor that is present in the wheal around the approximately 1400 nm wavelength range of the electromagnetic spectrum. SWIR images can be processed in grayscale with contrast enhancement. In some exemplary embodiments, a 640×512 pixel or other (higher or lower) resolution SWIR detector can be used to optimize imaging resolution and scannable surface area of the skin. In some embodiments, the SWIR detector 118 can include a 1400 nm bandpass filter to assist in obscuring veins in the skin while maintaining superior resolution and visibility of the wheal. An improved visualization of the wheal can therefore be achieved. In addition, the wheal of dark-skinned individuals can be more clearly defined using the SWIR detector 118 as opposed to visible light ranges.

Thermal detectors or cameras (e.g., long wavelength infrared (LWIR)) often use a microbolometer detector that absorbs infrared radiation in the, e.g., approximately 7,000 nm to approximately 14,000 nm, approximately 8,000 nm to approximately 14,000 nm, approximately 9,000 nm to approximately 14,000 nm, approximately 10,000 nm to approximately 14,000 nm, approximately 11,000 nm to approximately 14,000 nm, approximately 12,000 nm to approximately 14,000 nm, approximately 7,000 nm to approximately 13,000 nm, approximately 8,000 nm to approximately 12,000 nm, approximately 8,000 nm to approximately 13,000 nm, approximately 9,000 nm to approximately 12,000 nm, or the like, wavelength range.

Under normal or stable conditions, human skin generally emits thermal energy at approximately a 10,000 nm wavelength. Warmer regions of the skin, such as can occur from increased blood flow, can be detected by a thermal imaging camera. This is essential when measuring the area of a flare reaction that results in part from increased blood flow to the testing area 114. Improved visualization of the flare can therefore be achieved. In addition, thermal range detection of the flare allows for flare to be detected and analyzed in dark-skinned individuals, whereas flare detection through visible range imaging would be nearly impossible. In some embodiments, the thermal infrared detector can be incorporated into the device 102 to supplement the measurement and analysis of the SWIR detector 118. By capturing data from both the SWIR detector 118 and the thermal infrared detector, important data relating to the immuno-response of the patient can be gathered.

Still with reference to FIG. 1, in some embodiments, the device 102 can include a breakout box 126 (and/or a logic board). The breakout box 126 can be used to interconnect two or more components of the device 102. For example, the breakout box 126 can include one or more connectors for connecting the SWIR detector 118 and/or the detector 122. The breakout box 126 can further be used to integrate testing of signals received from the SWIR detector 118 and/or the detector 122, expedite maintenance, and streamline operation of the device 102 by simplifying access to the test signals.

In some embodiments, the device 102 can include a connection interface 128 (e.g., a universal serial bus (USB) connector, or the like) connected to the breakout box 126. The connection interface 128 can be used to interconnect a processing engine 130 with the breakout box 126 and the remaining components of the device 102. For example, images and/or data acquired by the SWIR detector 118 and/or the detector 122 can be transmitted via the breakout box 126 and the connection interface 128 to the processing engine 130. In some embodiments, the processing engine 130 can analyze the received data and can be configured to transmit results to the user interface 104. In some embodiments, the processing engine 130 can organize the received data and can be configured to transmit the data to the user interface 104 for further processing and analysis.

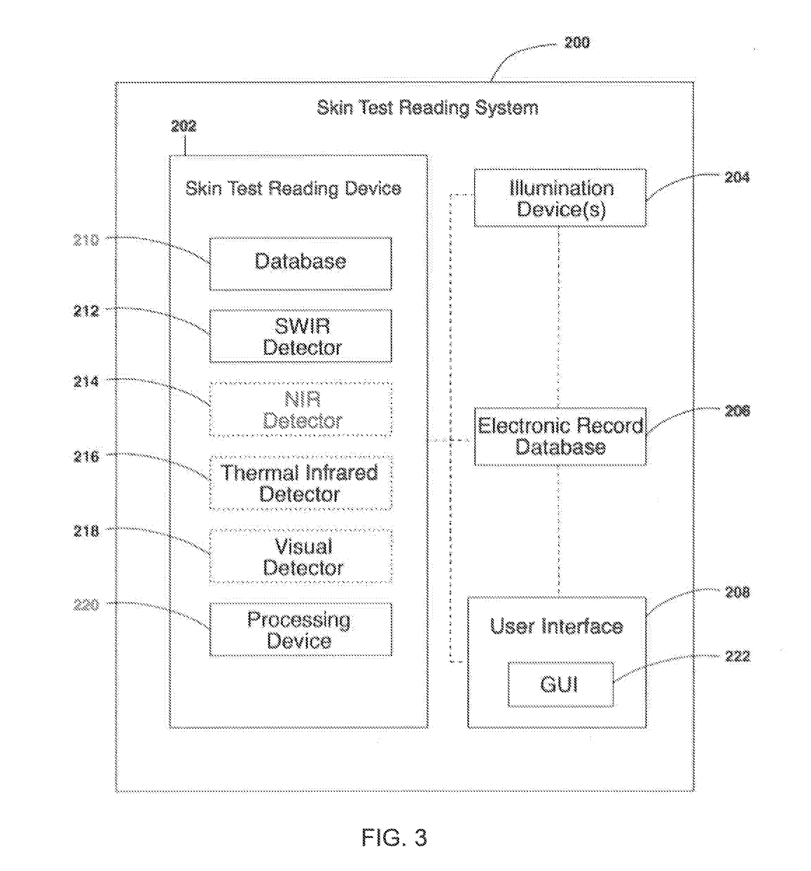

With reference to FIG. 3, a diagrammatic view of an exemplary skin test reading system 200 (hereinafter “system 200”) is provided. It should be noted that the system 200 can include one or more components of the system 100, and vice versa. The system 200 includes a skin test reading device 202 (hereinafter “device 202”) communicatively connected to one or more illumination devices 204, an electronic record database 206, and a user interface 208. As shown by the dashed lines, the device 202, illumination devices 204, electronic record database 206, and user interface 208 can be communicate (e.g., transmit/receive data) relative to each other.

The device 202 can include a database 210 disposed therein. The database 210 can be configured to electronically receive and store/organize a plurality of images and data captured by components of the device 202. The device 202 can include a SWIR detector 212. In some embodiments, the device 202 can include a NIR detector 214, a thermal infrared detector 216, and/or a visual detector 218. The device 202 can include a processing device 220 configured to programmatically receive and analyze data captured by one or more of the detectors 212-218 of the device 202. The processing device 220 can be configured to programmatically transmit data from the device 202 to one or more components of the system 200. For example, the processing device 220 can electronically transmit data from the device 202 to an electronic record database 206 (e.g., an electronic medical record database associated with a patient) for storage and/or further processing. As a further example, the processing device 220 can electronically transmit data from the device 202 to the user interface 208. The user interface 208 can include a GUI 222 for displaying the received data. In some embodiments, the processing device 220 can be configured to automatically capture one or more images with the detector(s) (e.g., upon sensing the placement of a testing area in a field-of-view of the detector(s)). In some embodiments, the processing device 220 can be configured to automatically analyze the captured image(s) and output the results to a graphical user interface.

The one or more illumination devices 204 can be used to illuminate the testing area 114. In particular, the illumination devices 204 can enhance operation and accuracy of the device 202 by enhancing the appearance of the wheal and/or flare. In some embodiments, the frequency of the illumination devices 204 can be tuned for optimal distribution and illumination over the testing area 114. In some embodiments, the illumination devices 204 can be tuned to specifically illuminate a wavelength range of interest (e.g., the SWIR range, the thermal infrared range, NIR or the like) to enhance reading of the wheal and/or flare. For example, soft dispersed lighting that emits wavelengths of radiation in the SWIR range can be used to properly illuminate the wheal during image capture by the SWIR detector. In some embodiments, the processing device 220 and/or the GUI 222 can be used to optimally tune the frequency of the illumination devices 204.

In some embodiments, the illumination devices 204 can be, e.g., light-emitting diodes (LEDs), tungsten incandescent bulbs, or the like, although alternative illumination devices 204 can be used. In some embodiments, the illumination devices 204 can provide radiation in the SWIR range that is invisible to the naked eye. In some embodiments, a flash bulb can be used such that constant radiation from the illumination device 204 (e.g., a tungsten source) does not increase surface temperature of the testing area of the skin. In particular, ambient temperatures of the testing area can be maintained substantially constant during testing since fluctuations in the temperature could impair the ability of the thermal infrared detector to identify flare responses on the skin.

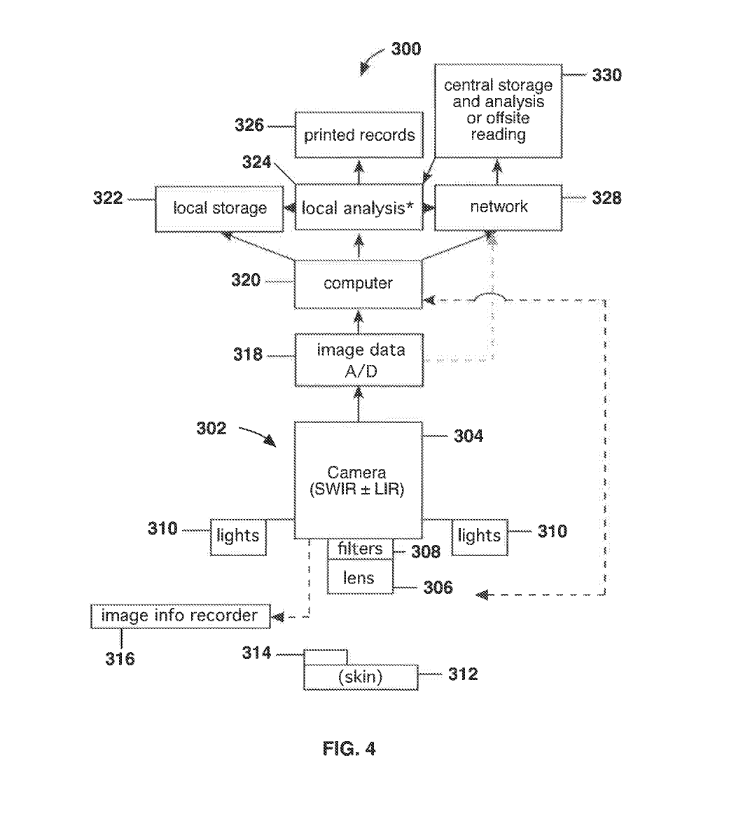

With reference to FIG. 4, a diagrammatic view of an exemplary skin test reading system 300 (hereinafter “system 300”) is provided. It should be noted that the system 300 can include one or more components of the system 100/200, and vice versa. The system 300 includes a skin test reading device 302 (hereinafter “device 302”). The device 302 can include an SWIR detector 304. The SWIR detector 304 can include a lens 306 and optionally includes one or more filters 308. The lens 306 can be optimized for skin test imaging and can include an auto-focusing function. In some embodiments, the auto-focus of the lens 308 can be regulated by the processing device 320 of the system 300. The filters 308 can be used to optimize the SWIR detector 304 for skin test imaging. For example, the filter 308 can be a 1400 nm bandpass filter to assist in obscuring veins in the skin while maintaining superior resolution and visibility of the wheal. In some embodiments, the device 302 can include a longwave infrared (LIR) detector, a NIR, a visual detector, a thermal infrared detector, or the like.

One or more illumination devices 310 can be operatively connected to the device 302 for illuminating a testing area 314 of skin 312 of a patient. The illumination devices 310 can optimize the SWIR detector 304 imaging for allergy skin testing. The system 300 can include a recording device 316 (e.g., an image or data recording device) communicatively connected to the device 302 and configured to receive image and/or data captured by the device 302 and relating to the testing area 314. In some embodiments, the recording device 316 can include a database of skin image patterns such that when data is received from the device 102, the recording device 316 can imprint or review the skin images to identify allergen patterns in the received data.

In some embodiments, the system 300 can include a device for imprinting skin images with information for identifying allergen patterns (e.g., laser imprinters, bar code imprinters, specialized skin safe markers, or the like). For example, the imprinter can assist in labeling each testing area of the patient to accurately identify the allergen being tested. In some embodiments, the imprinting device can be used to imprint skin images with information for registering and aligning the skin testing site relative to the detector(s). For example, the imprinting device can imprint markers on or around the testing area that can be identified by the detector(s) to center or align the position of the detector(s) relative to the testing site. In some embodiments, the imprint markers can assist the detector(s) in locating, centering on and/or zooming in on individual pricks of a skin prick test.

In some embodiments, the system 300 can include an auto-calibration function to calibrate the detector(s) of the device 302 relative to the testing area. For example, in some embodiments, the imprinting device can include an automated scale that measures wheal size and auto-calibrates the device 302. In some embodiments, the system 300 and/or device 302 can include an auto-calibration function separate from the imprinting device such that wheal and/or flare size can be calibrated to ensure accurate detection and measurement of wheal and/or flare size of a patient.

The system 300 can include an analog-to-digital converter 318 connected to the device 302. The converter 318 can convert the image or video stream from the SWIR detector 304 into a digital format. The system 300 can include a processing device 320 (e.g., a computer) configured to receive, analyze and manage the digital data received from the converter 318. The processing device 320 can include a GUI for displaying images and/or data to a user.

The system 300 can include a storage medium 322 (e.g., local storage) associated with the image management processing device 320. The storage medium 322 can store the data received by the processing device 320. The storage medium 322 can also include software algorithms for processing the image data. The system 300 can include an analysis engine 324 connected to the storage medium 322. The engine 324 can receive as input the image data from the device 302 and can programmatically execute the software algorithms to determine the immuno-response of the patient.

The processing device 320 and/or the engine 324 can be configured to process the received data and output a report or record 326 (e.g., a printed record, an electronic record, or the like). The record 326 can include a compilation of the relevant skin test information (e.g., images and test design) in a user-friendly manner for physically storing in a medical records storage. In some embodiments, the record 326 can be an electronic record for storing in an electronic medical records database. The system 300 can include a networking interface 328 for communicating with distributed networks associated with the system 300. For example, the networking interface 328 can permit the system 300 to communicate with data management devices outside of the system 300. In some embodiments, the system 300 can include a central storage database 330 for storing and analysis of the data.

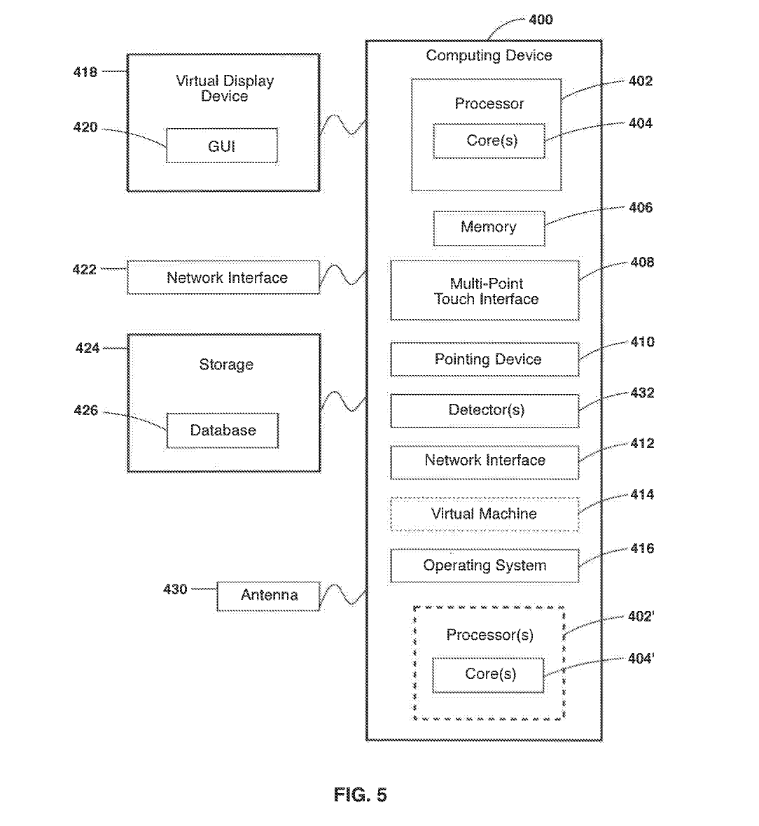

FIG. 5 is a block diagram of a computing device 400 configured to implement embodiments of the skin test reading systems in accordance with embodiments of the present disclosure. The computing device 300 can be a processing device or computer connected to the skin test reading devices. The computing device 400 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more flash drives), and the like. For example, memory 406 included in the computing device 400 may store computer-readable and computer-executable instructions or software for implementing exemplary embodiments of the present disclosure (e.g., the SWIR detector, alternative detectors, the illumination devices, transmission of captured data, processing of captured data, combinations thereof, or the like). The computing device 400 also includes a configurable and/or programmable processor 402 (including a central processing unit (CPU)) and associated core 404, and optionally, one or more additional configurable and/or programmable processor(s) 402′ and associated core(s) 404′ (for example, in the case of computer systems having multiple processors/cores), for executing computer-readable and computer-executable instructions or software stored in the memory 406 and other programs for controlling system hardware. Processor 402 and processor(s) 402′ may each be a single core processor or multiple core (404 and 404′) processor.

Virtualization may be employed in the computing device 400 so that infrastructure and resources in the computing device may be shared dynamically. A virtual machine 414 may be provided to handle a process running on multiple processors so that the process appears to be using only one computing resource rather than multiple computing resources. Multiple virtual machines may also be used with one processor.

Memory 406 may include a computer system memory or random access memory, such as DRAM, SRAM, EDO RAM, and the like. Memory 406 may include other types of memory as well, or combinations thereof.

A user may interact with the computing device 400 through a visual display device 418, such as a computer monitor, which may display one or more user interfaces 420 that may be provided in accordance with exemplary embodiments (e.g., the user interface 208 associated with the GUI 222). The computing device 400 may include other I/O devices for receiving input from a user, for example, a keyboard, one or more detectors 432 (e.g., a SWIR detector, NIR detector, visual detector, thermal infrared detector, or the like), or any suitable multi-point touch interface 408, a pointing device 410 (e.g., a mouse), or the like. The keyboard 408 and the pointing device 410 may be coupled to the visual display device 418. The computing device 400 may include other suitable conventional I/O peripherals.

The computing device 400 may also include one or more storage devices 424, such as a hard-drive, CD-ROM, or other computer readable media, for storing data and computer-readable instructions and/or software that implement exemplary embodiments of the skin test reading systems described herein. In some embodiments, the storage device 424 can store images and/or data captured by the detectors 432. Exemplary storage device 424 may also store one or more databases 426 for storing any suitable information required to implement exemplary embodiments. For example, exemplary storage device 424 can store one or more databases 426 for storing information, such as data stored within the database 210, 206, and computer-readable instructions and/or software that implement exemplary embodiments described herein. The databases 426 may be updated manually or automatically at any suitable time to add, delete, and/or update one or more items in the databases 426.

The computing device 400 can include a network interface 412 configured to interface via one or more network devices 422 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 412 may include a built-in network adapter, network interface card, PCMCIA network card, card bus network adapter, wireless network adapter, USB network adapter, modem or any other device suitable for interfacing the computing device 400 to any type of network capable of communication and performing the operations described herein. The computing device 400 can also include one or more antennas 430 for wirelessly interfacing the computing device 400 to any type of wireless network communication protocol and performing the operations described herein. Moreover, the computing device 400 may be any computer system, such as a workstation, desktop computer, server, laptop, handheld computer, tablet computer (e.g., the iPad™ tablet computer), mobile computing or communication device (e.g., the iPhone™ communication device), or other form of computing or telecommunications device that is capable of communication and that has sufficient processor power and memory capacity to perform the operations described herein.

The computing device 400 may run any operating system 416, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, or any other operating system capable of running on the computing device and performing the operations described herein. In exemplary embodiments, the operating system 416 may be run in native mode or emulated mode. In an exemplary embodiment, the operating system 416 may be run on one or more cloud machine instances.

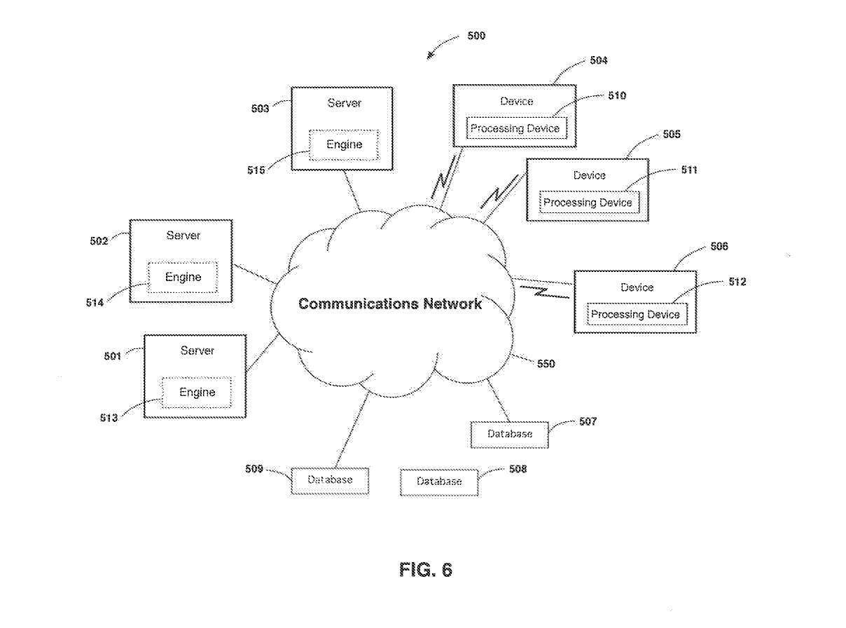

FIG. 6 is a block diagram of a distributed environment 500 for implementing embodiments of the skin test reading system in accordance with embodiments of the present disclosure. In particular, the distributed environment 500 can be used to facilitate communication between a plurality of skin test reading devices such that captured data can be electronically stored in electronic medical records distributed over a network. The environment 500 can include servers 501-503 operatively coupled to one or more devices 504-506 including processing devices 510-512, respectively, and databases 507-509, via a communication network 550, which can be any network over which information can be transmitted between the servers 501-503, devices 510-512, and databases 507-509. For example, the communication network 550 can be the Internet, Intranet, virtual private network (VPN), wide area network (WAN), local area network (LAN), and the like. Those skilled in the art will recognize that the databases 506-509 can be incorporated into one or more of the servers 501-503 such that one or more of the servers 501-503 can include the databases 506-509.

In some embodiments, the databases 506-509 can store information relating to the images and/or data captured and analyzed by the devices 504-506. In some embodiments, information relating to the captured images and/or data can be distributed over one or more of the databases 506-509.

In some embodiments, embodiments of the servers 501-503 can be configured to implement one or more portions of engines 513-515 associated with the devices 504-506. The engines 513-515 can be configured to programmatically analyze the captured data from the devices 504-506 and output the results of the allergy skin test. In some embodiments, the one or more engines 513-515 can be implemented in a distributed configuration over the servers 501-503.

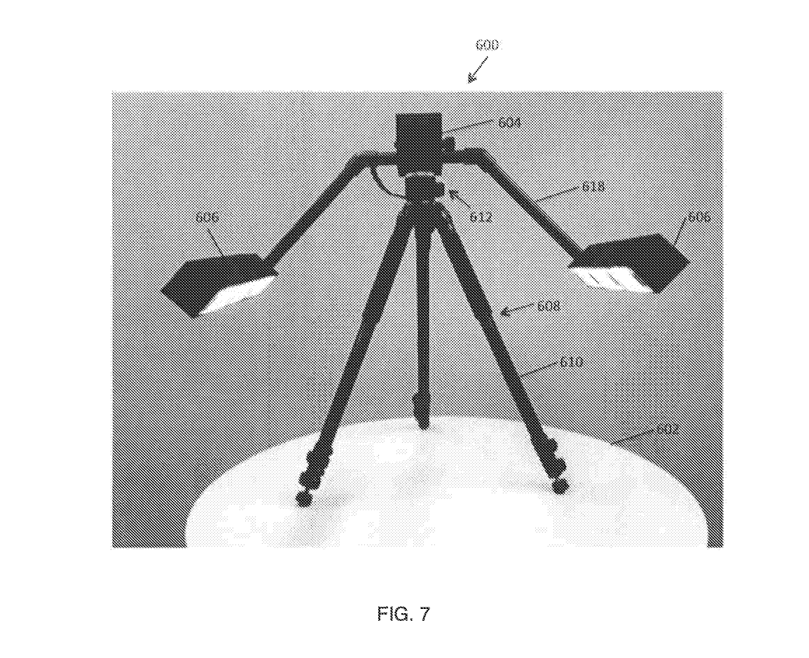

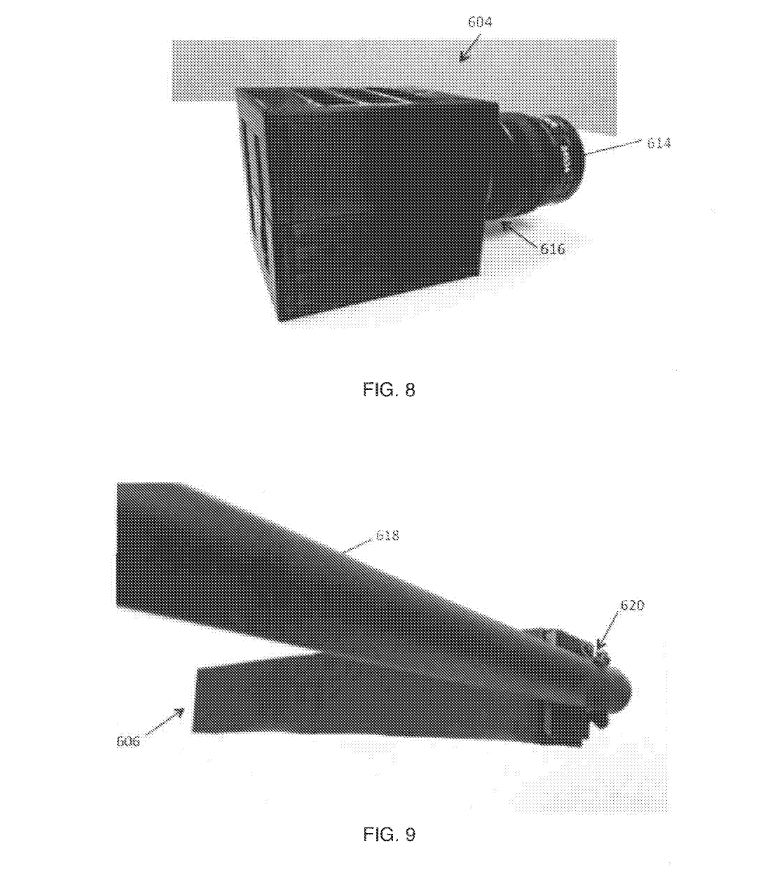

With reference to FIGS. 7-9, perspective and detailed views of an exemplary prototype 600 of a skin test reading device are provided. In particular, FIG. 7 shows the prototype 600 on a platform 602, FIG. 8 shows a detailed view of an SWIR detector 604, and FIG. 9 shows a detailed view of an illumination device 606. The platform 602 can be configured to support at least a portion of a patient, e.g., an arm, such that the testing area of the patient can be positioned under the SWIR detector 604. In some embodiments, the SWIR detector 604 can be positioned approximately five feet or less from the testing area of the patient. In some situations, large surface areas of skin which need to be captured with multiple distinct images can be assembled in a mosaic pattern using software and/or hardware components such that an overall image of the testing area can be obtained.

The skin test reading device can include a supporting structure 608, e.g., a tripod, or the like, with three supporting legs 610. The legs 610 can be telescoping such that the position of the SWIR detector 604 relative to the testing area of the patient can be adjusted. The supporting structure 608 can further include an interlocking mechanism 612 configured and dimensioned to mechanically interlock relative to a complementary interlocking mechanism of the SWIR detector 604 such that the SWIR detector 604 can be detachably mounted to the supporting structure 608 over the platform 602. In some embodiments, the interlocking mechanism 612 can be adjusted to allow for rotation of the SWIR detector 604 relative to the supporting structure 608 such that the tilt angle of the SWIR detector 604 can be adjusted relative to the testing area. Although FIGS. 6-8 show a supporting structure 608 in the form of a tripod, in some embodiments, the devices discussed herein can be, e.g., handheld such that the device can be supported by the practitioner and the position/orientation of the device is regulated by a practitioner, supported by a mobile structure on wheels such that the device can be transported to the desired location relative to the patient, supported by a swing arm attached to a wall that allows for multiple degrees of freedom for positioning the device relative to the patient, or the like.

As discussed above, the SWIR detector 604 can include a lens 614 and a filter 616. The supporting structure 608 can include one or more arms 618 extending outwardly therefrom. In some embodiments, the arms 618 can be hingedly connected to the supporting structure 608 such that the position of the arms 618 can be adjusted. The arms 618 can be configured and dimensioned to support one or more illumination devices 606 over the platform 602. The arms 618 can include an interlocking mechanism 620 for hingedly connecting the illumination device 606 to the respective arm 618. The tilt angle or orientation of the illumination device 606 relative to the platform 602 can therefore be adjusted based on the illumination needs of the user. In some embodiments, rather than or in combination with the illumination devices 606, the system or device can include, e.g., a light ring (with or without a parabolic reflector) incorporated or attached to the detector(s), one or more light boxes, or the like, for illuminating the testing area.

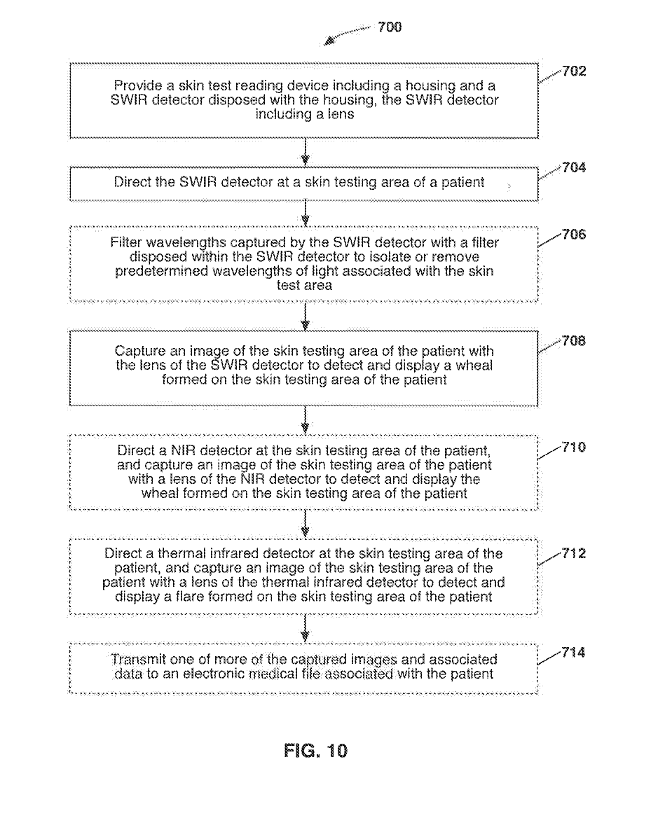

With reference to FIG. 10, an exemplary automated process 700 of reading an allergen skin test with the exemplary skin test reading devices discussed herein is provided. Although illustrated as an automated process 700, in some embodiments, one or more steps of the process 700 can be performed manually. Initially, at step 702, a skin test reading device can be provided that includes a housing and a SWIR detector disposed within the housing. The SWIR detector can include a lens configured to capture one or more images of the skin testing area. At step 704, the SWIR detector can be directed or oriented at the skin testing area of the patient such that the field-of-view of the SWIR detector captures the desired portion of the skin testing area. In some embodiments, the field-of-view of the SWIR detector (or alternative detectors) can be adjusted by zooming in or out to capture, e.g., the entire body of the patient, the entire arm of the patient, the entire testing area of the patient, a group of individual pricks in the testing area, an individual prick in the testing area, or the like. In some embodiments, at step 706, a filter disposed within the SWIR detector can be used to isolate or remove (e.g., filter) predetermined wavelengths of light associated with the skin testing area that are captured by the SWIR detector. At step 708, an image of the skin testing area of the patient can be captured with the lens of the SWIR detector to detect and display a wheal formed on the skin testing area of the patient.

In some embodiments, at step 710, a NIR detector can also be directed or oriented at the skin testing area of the patient. The NIR detector can capture an image of the skin testing area of the patient with a lens to detect the wheal formed on the skin testing area of the patient. In some embodiments, at step 712, a thermal infrared detector can be directed or oriented at the skin testing area of the patient. The thermal infrared detector can capture an image of the skin testing area of the patient with a lens to detect a flare formed on the skin testing area of the patient. The flare can be associated with the wheal detected by the SWIR detector. In some embodiments, at step 714, one or more of the captured images and associated data can be electronically transmitted to an electronic medical file or storage associated with the patient and/or a research/hospital facility. In some embodiments, distinct images generated from adjacent detectors can be combined, overlaid and/or fused together for processing and/or storage.

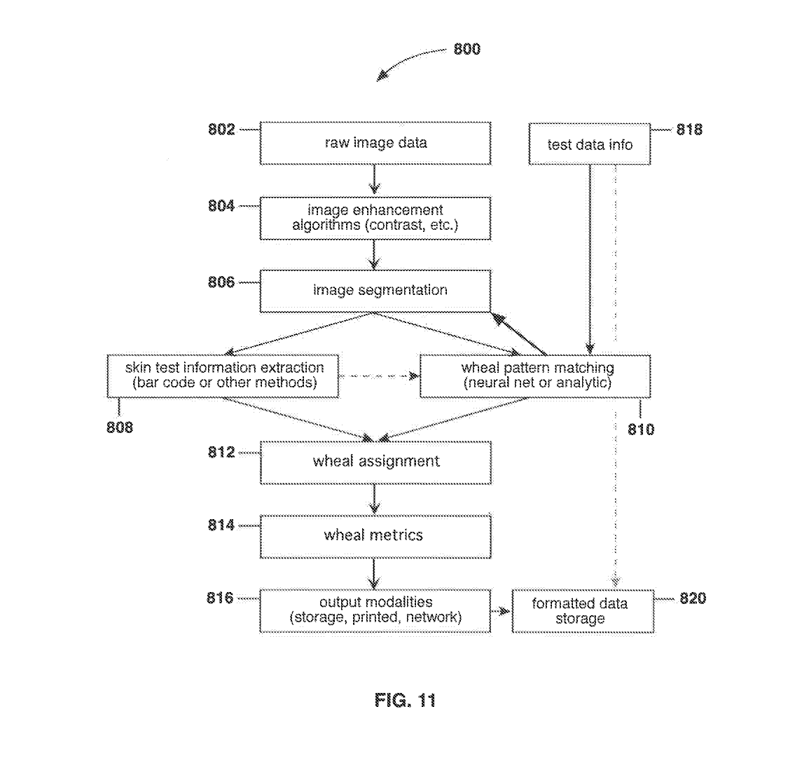

With reference to FIG. 11, an exemplary automated process 800 of reading and/or processing an allergen skin test with the exemplary skin test reading devices discussed herein is provided. Although illustrated as an automated process 800, in some embodiments, one or more steps of the process 800 can be performed manually. After one or more images have been captured by the skin test reading device, at step 802, raw images can be transmitted into a processing device or computer by an analog-to-digital converter or interface. At step 804, the image data can be processed to enhance the image and optimize subsequent processing. For example, image enhancement algorithms can be programmatically executed by a processing device to enhance the color, contrast, brightness, sharpness, combinations thereof, or the like, of the image data. At step 806, segmentation of the image data can be performed to separate the image components into skin test results and the embedded skin test information.

At step 808, the skin test information can be extracted for further analysis (e.g., matching, scaling, measuring, quantifying, or the like). The extracted data can be transmitted to a processing device for analysis. At step 810, the processing device can be used to analyze the skin test information to identify wheals and/or flares. In some embodiments, the system can include a library or database of wheal and/or flare patterns previously collected and organized. The wheal and/or flare patterns can be used in an image matching algorithm to match the stored wheal and/or flare patterns with the wheal detected during the skin test. The image matching process can be performed in an iterative manner to optimize identification of the wheal and/or flare. In some embodiments, at step 818, basic patient information and/or test data information can be introduced into the pattern matching algorithm for association of the results with a particular patient.

At step 812, the detected and analyzed wheal can be assigned to a specific allergen. In particular, the extracted skin test information can be combined with the wheal and/or flare identifications to assign the results to proper allergens. Thus, an association can be created between the skin test information, the wheal and/or flare identification information, and an allergen. At step 814, an analysis of the wheal and/or flare segmented image data can be performed for standard metrics of the skin test. For example, the extent or size of the wheal and/or flare can be determined based on the captured images and/or data. At step 816, the analyzed data can be processed for outputting in the form of a report. For example, the report can be output to storage, printed records, a network system (e.g., an electronic medical record), an offsite facility (e.g., a hospital, a research laboratory, or the like), combinations thereof, or the like. In some embodiments, at step 820, the basic patient information and the associated test results can be combined, processed and stored in a database for future access. In some embodiments, the database can be a physical and/or a cloud-based data management system for storing data for future retrieval.

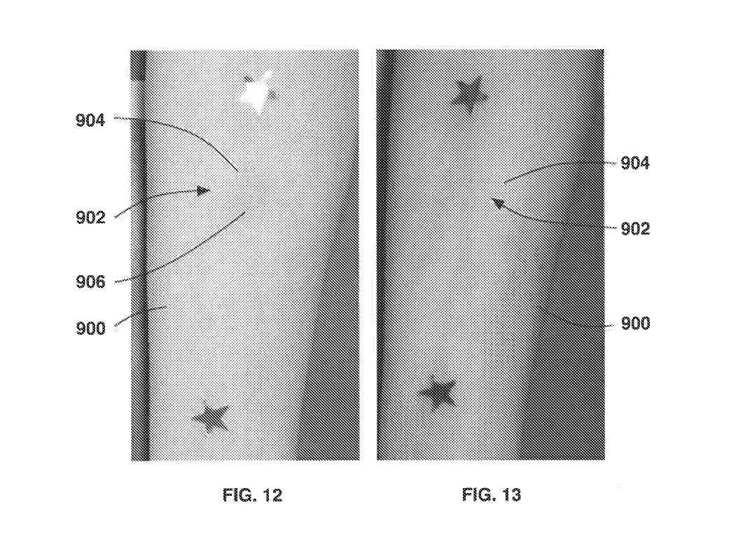

With reference to FIGS. 12-15, images of a skin testing area of a patient captured by a visual detector, NIR detector, thermal infrared detector, and SWIR detector, respectively, are provided. In particular, the images illustrate the differences in visibility of the wheal and/or flare formed on the skin testing area and emphasize the advantageous properties of implementing the thermal infrared detector and/or the SWIR detector in the disclosed skin test reading devices. In some embodiments, the upper and lower bounds of a testing area can be marked (e.g., by stars or other elements placed on the patient) such that the detector(s) visualize the markings and scan only the bounded area.

With reference to FIG. 12, the image captured by a visual wavelength detector is provided. The image shows a forearm 900 of the patient with a testing area 902. Due to the visual wavelength used, the wheal 904 and surrounding flare 906 are only slightly visible and it would be difficult to determine the size or extent of the wheal 904 and flare 906 for analysis. With reference to FIG. 13, the image captured of the same patient and testing area 902 by the NIR detector is provided. Although the visualization of the veins is improved, the wheal 904 is only slightly visible and the flare 906 is not visible. Thus, it would be difficult to determine the size or extent of the wheal 904 and flare 906 for analysis.

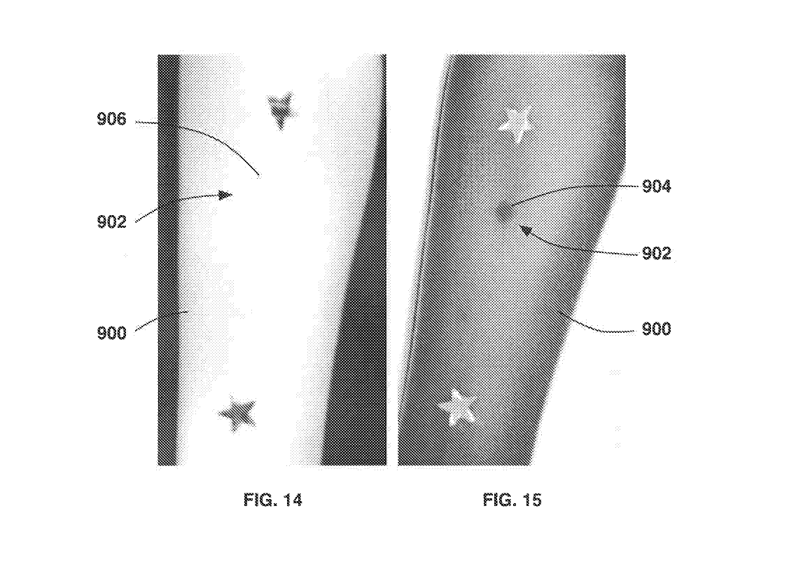

With reference to FIG. 14, the image captured of the same patient and testing area 902 by the thermal infrared detector is provided. Although the wheal 904 is not visible in this image, the flare 906 is clearly visible. Thus, the image captured by the thermal infrared detector can be used to accurately measure and determine the flare 906 resulting from an allergen. With reference to FIG. 15, the image captured of the same patient and testing area 902 by the SWIR detector is provided. Although the flare 906 is not visible in this image, the wheal 904 is clearly visible. In particular, the perimeter of the wheal 904 is clearly shown, preventing potential errors in incorrectly determining and measuring the extent of the wheal 904 (irrespective of the darkness of the skin of the patient). Thus, the image captured by the SWIR detector can be used to accurately measure and determine the wheal 904 resulting from an allergen. In some embodiments, the image captured by the SWIR detector can be used to determine information relating to the wheal 904, and the image captured by the thermal infrared detector can be used to supplement the data gathered from the SWIR detector image by providing information relating to the flare 906.

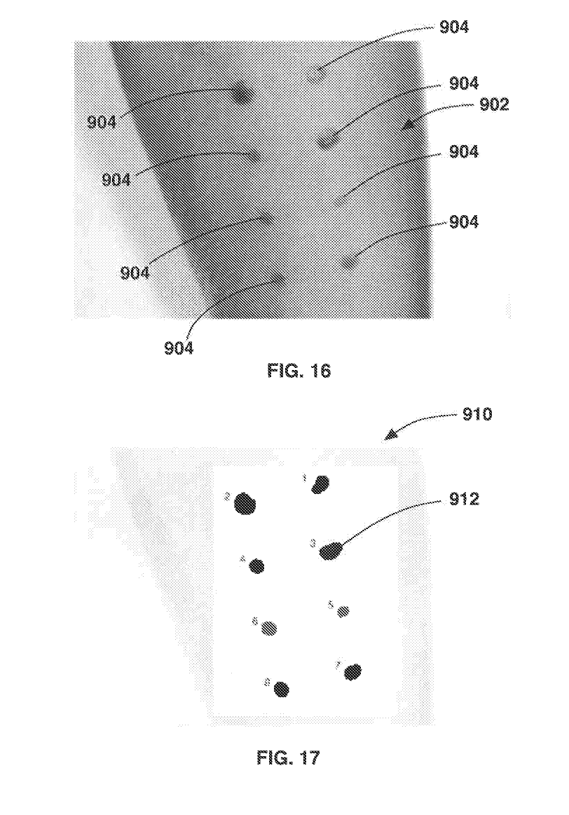

In some embodiments, the systems discussed herein can process the image data captured by the detector(s) and analyze the data to output a visualization of the identified wheal and/or flare, as well as data associated with the wheal and/or flare. For example, FIG. 16 shows an image of a testing area 902 of a patient captured by a SWIR detector with multiple wheals 904 visualized and formed thereon. Although visualization of the wheals 904 is clear due to the SWIR detector functionality, further analysis of the SWIR image can be performed.

FIG. 17 shows a processed image 910 of the testing area 902 with each wheal 904 represented by a pixilated wheal representation 912. In some embodiments, the wheal representation 912 can be provided against a white background to provide greater visualization of the wheal dimension and configuration. The processed image 910 can provide labeling for each of the wheal representations 912 (e.g., numbers 1-8) such that each wheal 904 can be independently identifiable. In addition to the visual representation, the system can provide a table of metrics associated with the testing area 902. An exemplary Table 1 is provided below. It should be understood that a substantially similar image and/or table can be provided for flare information obtained from a thermal infrared detector image.

TABLE 1
Wheal Metrics

| | | Wheal Area | Wheal Major | Wheal Minor |
|---|---|---|---|---|
| Wheal No. | Allergen | (mm²) | Axis (mm) | Axis (mm) |
| 1 | A | 112 | 13.9 | 9.1 |
| 2 | B | 191 | 16.7 | 14.0 |
| 3 | C | 155 | 17.0 | 11.2 |
| 4 | D | 86 | 10.6 | 9.0 |
| 5 | E | 47 | 7.5 | 6.4 |
| 6 | F | 88 | 10.8 | 9.5 |
| 7 | G | 105 | 12.1 | 10.4 |
| 8 | H | 94 | 11.2 | 9.8 |

The generated table can list a wheal number associated with each wheal 904, an allergen tested at the wheal 904, a wheal area, a wheal major axis, and a wheal minor axis. In particular, the system can process and determine the area of each wheal 904 such that estimating the wheal 904 size is no longer necessary. An accurate determination of the effect of an allergen can therefore be determined based on the provided metrics. The wheal major and minor axis can represent the detected position of each wheal 904 relative to a default location of the testing area 902.

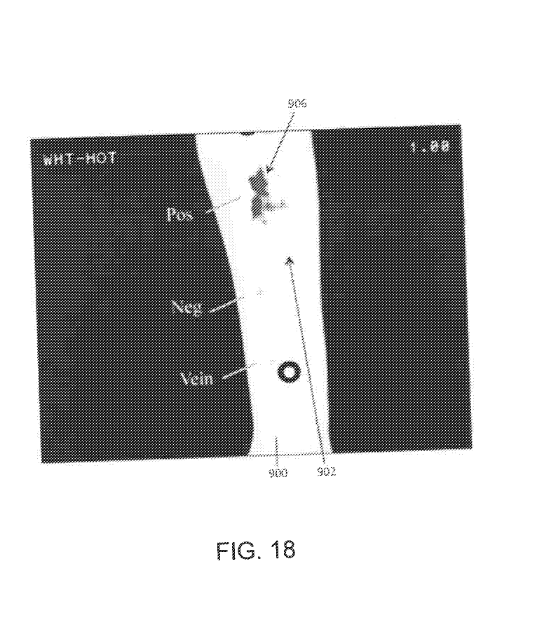

With reference to FIG. 18, an image captured by a thermal infrared detector is provided. In particular, the thermal infrared detector can provide a clear visualization of the flare 906 resulting from introduction of an allergen to the testing area 902. As shown in FIG. 18, during the allergen skin test, a negative (“Neg”) puncture test was performed with a compound that was known not to induce a wheal or flare, and a positive (“Pos”) puncture test was performed with a compound that was known to induce a wheal and/or flare. Based on the image of FIG. 18, the flare 906 formed at the positive puncture test area due to the compound used, while a flare is not visible in the negative positive puncture test area. The system can therefore focus the analysis on the flare 906. Similarly, although the image shows a portion of the vein of the patient, based on the position of the testing area 902 and the amount of visible flare, the system can focus the analysis on the flare 906 resulting from the allergen and can disregard the additional vein data.

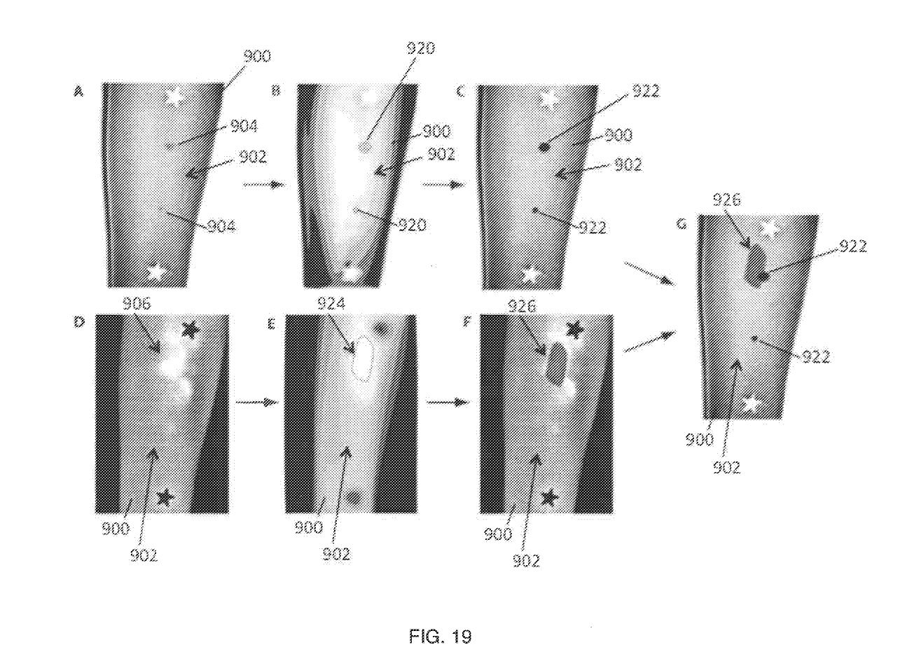

With reference to FIGS. 19A-G and as noted above, in some embodiments, the image captured by the SWIR detector can be supplemented with the image captured by the thermal infrared detector. The data from the SWIR detector and the thermal infrared detector can be combined (e.g., overlaid) to create a composite image that provides a clear visualization to the practitioner of the wheal and flare data associated with an allergen skin test.

For example, in FIG. 19A shows an unaltered SWIR detector image of a testing area 902 with two wheals 904 (i.e., positive and negative control) formed thereon. In particular, the upper wheal 904 corresponds to the positive control, while the lower wheal corresponds to the negative control. In FIG. 19B, a live trace function tool can be executed in a graphics software program to process the SWIR image of FIG. 19A. The processed image can provide tracings 920 around each wheal 904. In particular, the tracings 920 can represent the extent and perimeter of the wheal 904. In FIG. 19C, the tracings 920 can be filled in with a solid color to create wheal representations 922. The wheal representations 922 can be superimposed onto the original image of FIG. 19A to provide an improved visualization of the wheals 904.

In FIG. 19D, an unaltered thermal infrared detector image of the testing area 902 is provided. As can be seen, a flare 906 formed around the upper wheal 904 and no flare 906 has formed around the lower wheal 904. In FIG. 19E, the live trace function tool of the graphics software program can process the thermal infrared image of FIG. 19D. The processed image can provide a tracing 924 around the flare 906. In particular, the tracing 924 can represent the extent and perimeter of the flare 906. In some embodiments, a smoothing algorithm can be executed to generate the smooth tracing 924 (e.g., a polygon structure) resulting in an intensity mapping function with smoothing, thereby obtaining an accurate representation of the area of the flare 906.

In FIG. 19F, the tracings 924 can be filled in with a solid color to create a flare representation 926. In FIG. 19G, the SWIR image of FIG. 19C and the thermal infrared image of FIG. 19F can be superimposed over the original image of the testing area to yield a clear digital representation of the wheal and flare reaction. The color of the flare representation 926 can differ from the color of the wheal representation 922 such that the two representations 922, 926 can be visualized even in an overlapped orientation. The resulting composite image can be used to more effectively and accurately generate quantifiable measurements for the allergen skin test in a time-efficient manner.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A skin test reading device, comprising:
a short wave infrared (SWIR) detector including a lens configured to enable the SWIR detector to capture an image of a skin testing area of a patient;
wherein the SWIR detector is configured to detect a wheal formed on the skin testing area of the patient.

2. The skin test reading device of claim 1, wherein the SWIR detector is configured to detect the wheal formed on the skin testing area of the patient during an allergen test.

3. The skin test reading device of claim 1, wherein the SWIR detector comprises a filter configured to isolate or remove predetermined wavelengths of light associated with the skin testing area.

4. The skin test reading device of claim 3, wherein the filter is configured to obscure veins in the testing area to enhance detection and visibility of the wheal formed on the skin testing area of the patient.

5. The skin test reading device of claim 3, wherein the SWIR detector is configured to detect wavelengths between 1,000 nm and 3,000 nm.

6. The skin test reading device of claim 1, further comprising a near wave infrared (NIR) detector including a lens configured to enable the NIR detector to capture an image of the wheal formed on the skin testing area of the patient.

7. The skin test reading device of claim 6, wherein the NIR detector is configured to detect wavelengths between 700 nm and 1,000 nm.

8. The skin test reading device of claim 1, further comprising a thermal infrared detector including a lens configured to enable the thermal infrared detector to capture an image of the skin testing area of the patient.

9. The skin test reading device of claim 8, wherein the thermal infrared detector is configured to detect a flare formed on the skin testing area of the patient during an allergen test.

10. The skin test reading device of claim 9, wherein the thermal infrared detector is configured to detect wavelengths between 7,000 nm and 14,000 nm.

11. The skin test reading device of claim 1, further comprising a visual wavelength detector including a lens configured to enable the visual wavelength detector to capture an image of the skin testing area of the patient.

12. The skin test reading device of claim 1, further comprising a processing device configured to analyze the wheal in the captured image of the skin testing area of the patient and output a dimension associated with the wheal.

13. The skin test reading device of claim 12, wherein the dimension associated with the wheal is a wheal diameter.

14. The skin test reading device of claim 1, further comprising a graphical user interface configured to display the captured image of the skin testing area of the patient with the wheal.

15. A method of reading a skin test, comprising:
providing a skin test reading device, the skin test reading device including a short wave infrared (SWIR) detector including a lens;
directing the SWIR detector at a skin testing area of a patient; and
capturing an image of the skin testing area of the patient with the SWIR detector;
wherein the SWIR detector is configured to detect a wheal formed on the skin testing area of the patient.

16. The method of claim 15, further comprising the step of filtering wavelengths captured by the SWIR detector with a filter disposed within the SWIR detector to isolate or remove predetermined wavelengths of light associated with the skin testing area.

17. The method of claim 15, further comprising the step of directing a near wave infrared (NIR) detector at the skin testing area of the patient, and capturing an image of the skin testing area of the patient with the NIR detector, the NIR detector being configured to detect the wheal formed on the skin testing area of the patient.

18. The method of claim 15, further comprising the step of directing a thermal infrared detector at the skin testing area of the patient, and capturing an image of the skin testing area of the patient with the thermal infrared detector, the thermal infrared detector being configured to detect a flare formed on the skin testing area of the patient.

19. The method of claim 15, further comprising the step of transmitting the image captured by the SWIR detector to an electronic medical file.

20. A skin test reading system, comprising:
a skin test reading device including (i) a short wave infrared (SWIR) detector including a lens configured to enable the SWIR detector to capture an image of a skin testing area of a patient, wherein the SWIR detector is configured to detect a wheal formed on the skin testing area of the patient;
a supporting structure configured to mount the SWIR detector over the skin testing area of the patient;
an illumination device mounted to the supporting structure and configured to illuminate at least a portion of the skin testing area of the patient; and
a graphical user interface configured to display the image captured by the SWIR detector.
